(19) United States
(12) Patent Application Publication
(10) Pub. No.: US 2017/0209618 A1
(43) Pub. Date: Jul. 27, 2017

(54) COMPOSITE ePTFE AND SILICONE SOFT TISSUE IMPLANTS TO MINIMIZE CAPSULAR CONTRACTURE, WEIGHT, INFECTION AND PALPABILITY

(71) Applicant: Mark A. Anton, Newport Beach, CA (US)

(72) Inventor: Mark A. Anton, Newport Beach, CA (US)

(21) Appl. No.: 15/402,199

(22) Filed: Jan. 9, 2017

Related U.S. Application Data
(60) Provisional application No. 62/276,661, filed on Jan. 8, 2016.

(51) Int. Cl. A61L 27/16; A61L 27/54; A61L 27/56; A61F 2/12; A61L 27/18

(52) U.S. Cl. CPC A61L 27/16; A61F 2/12; A61L 27/18; A61L 27/56; A61L 27/54; A61F 2310/00389; A61L 2300/414; A61L 2430/34

(57) ABSTRACT

Methods, systems, apparatuses and devices for implantation in a soft-tissue biological environment that include a primary layer for containing a filler substance, an interface and a secondary layer, including embodiments where the secondary layer an ePTFE layer, the primary layer is a silicone layer, the interface is mechanical or adhesive and the filler substance can include particulates and lattices.

### CROSS-REFERENCE TO RELATED APPLICATIONS

The present application claims priority to U.S. Provisional Application No. 62/276,661 entitled “GORE-TEX COVERED BREAST IMPLANTS TO MINIMIZE CAPSULAR REACTION, IMPLANT WEIGHT AND INFECTION WHILE REDUCING PALPABILITY” filed Jan. 8, 2016 which is hereby incorporated by reference in its entirety. This application is related to U.S. Provisional Application No. 62/060,480, entitled “GORETEX COVERED BREAST IMPLANTS TO MINIMIZE CAPSULAR REACTION” filed Oct. 6, 2014; U.S. Provisional Application No. 62/066,704, entitled “EPTFE IMPLANTS” filed Oct. 21, 2014; and U.S. Non-Provisional application Ser. No. 14/876,754, entitled “GORE-TEX COVERED BREAST IMPLANTS TO MINIMIZE CAPSULAR REACTION AND INFECTION WHILE REDUCING PALPABILITY” filed Oct. 6, 2015, each of which are hereby incorporated by reference in their entirety.

### BACKGROUND OF THE INVENTION

Plastic surgeons perform procedures to place implants throughout the body more than all other medical specialties. Implants can be used for one or more purposes and can be categorized based on various aspects of their medical purpose, function, design aesthetics or implantation location, geolocation tracking ability, neural identification and various others.

Aesthetic implants are used to modify, improve or otherwise alter cosmetic features of the face and other contours related to other body parts. As such, they can be considered to be an enhancement that simulates the look and feel of hard or soft tissues. Medical implants can be used for medical treatment purposes such as reconstruction of injured or destroyed tissue, electrical impulse delivery, delivery of substances such as drugs and monitoring of bodily functions.

Facial implants are generally placed in a specific location to enhance and simulate hard tissues like bone and cartilage. Examples of facial implants include cheek, nose and chin implants. Implants for body locations such as breasts, pectorals, buttocks and calves are placed to enhance or simulate soft tissues. The most common body implants are breast implants. Breast implants can be used for cosmetic breast augmentation and breast reconstruction after cancer or other surgeries. Breast implants can be used to change the size, firmness, shape, location or orientation of the breast.

During a surgical procedure, a breast implant is implanted in the chest area. After the procedure occurs, a collagen scar capsule typically forms around the breast implant. Ideally, the capsule should retain the shape of the implant while being slightly larger than the implant itself. However, as part of the healing process from the surgical procedure, capsules are known to contract in varying amounts. This process is called capsular contracture.

In some instances, capsular contracture can be a desired effect. Examples of desired effects include implantations where the implant should appear and feel like bone, such as cheek and chin implants. In other instances, it may be highly undesirable for implants to be firm or hard, such as where an implant is meant to simulate the appearance and feel of soft tissue.

Capsular contracture can result in the capsule shrinking or contracting to such a degree that it grips and compresses the implant. This can cause a large increase in pressure on the implant and the implant will then physically feel unnaturally firm and even hard to the outside touch. Secondary effects of capsular contracture can include significant breast shape distortion, pain for the subject resulting from the shrinking capsule pulling on surrounding sensory nerves and reduction in the efficacy of mammograms. Capsular contracture currently occurs at a variable rate of between 15% and 45% after implantation procedures.

The likelihood of capsular contracture can be reduced by various mechanisms. One of these mechanisms is placement of the breast implant in a sub-muscular position. Most current breast implantation procedures place an upper half of the breast implant in a location underneath a pectoralis muscle, since this location has been associated with a significant reduction in the likelihood of capsular contracture.

Another mechanism to reduce the likelihood of capsular contracture is to manipulate the breast implant during the manufacturing process. Current breast implants are often a silicone rubber shells filled with silicone gel, saline or other biologically safe filler substance. An outer surface of the silicone rubber shell can be manufactured as smooth or textured in a variety of ways. Implants having smooth outer surfaces are used to stimulate a surrounding smooth scar or collagen capsule to form. This capsule ideally stays larger than the implants, such that the implant can be free to move within the capsule. This movement simulates a more natural appearance while also enhancing the breast. Implants having textured outer surfaces induce and perform different bodily reactions, as discussed elsewhere herein.

Previously contemplated methods regarding breast implant devices include using different types of materials and outer coatings of the breast implants. Materials that have been considered include Gore-Tex, expanded polytetrafluoroethylene (ePTFE) and Teflon. These materials have not been widely implemented for various reasons as described in the prior art but are likely to have many beneficial properties. These reasons are variously described, as follows:

Previous patents that have used ePTFE as an outer layer coating have generally done so in order to create textured surfaces that try to encourage capsular tissue ingrowth from surrounding tissues. U.S. Pat. Nos. 8,647,393; 8,372,423; and 5,779,734 which are incorporated herein in their entirety by reference are examples of such ingrowth embodiments. As described in the stated goal of the U.S. Pat. No. 8,647,393 patent: “[d]isclosed herein are implantable devices coated with microporous surface layers with macrotopographic features that improve bio-integration at the interface of the implantable devices and the surrounding tissue.”

A recent non-patent reference states that “[a]fter multiple experimental and clinical trials, there seems to be a strong consensus that the use of textured outer shell surfaces, in comparison with smooth surfaces, is able to decrease the incidence of capsular contracture by disrupting contractile forces around the implant, emphasizing the need for better physical properties than cellular or pharmacological strategies of contracture formation.” Capsular Contracture by silicone breast implants: possible causes, biocompatibility, and prophylactic strategies, Steiert AE et al, Med Devices (Auckl) 2013; 6:211-218, which is hereby incorporated in its entirety by reference.

Another paper, published in November 1963 and titled Teflon-Silicone Implants by Benjamin Edwards, MD described the use of “Teflon felt” over a silicone implant because “[t]he following characteristics of an ideal breast implant are well known to every plastic surgeon. Such an implant . . . should become firmly attached to body tissues.” Plastic and Reconstructive Surgery Nov 1963; Vol. 32, No. 5, pp 519-526, which is hereby incorporated in its entirety by reference. The author further stated that in his opinion “[i]t is most important to have some sort of rough surface on the outside . . . As we have used here, a layer of Teflon foam.”

Although there are a number of plastic surgery journal articles that have suggested that textured implants are preferable to smooth surfaced implants, there remains a great deal of confusion and differing opinions regarding textured implants. In the past, the majority of surgeons and implant manufacturing companies believed that it was important and desirable to cause capsular tissues to adhere to and integrate with the outer surface of the textured implant. However, currently the majority consensus is that a majority of breast implants should not have surrounding capsular tissue adhering to the outer surface of a textured implant.

In addition, recent studies have shown that BIA-ALCL (Breast Implant Associated-Anaplastic Large Cell Lymphoma) has a much higher incidence in textured implants than smooth surfaced implants. This might well be related to the effects of chronic mechanical irritation caused by the rough outer surface of the implant on the surrounding unattached capsular tissue planes. There have been no known cases of BIA-ALCL in patients with only smooth surfaced implants.

Breast implants with textured surfaces have also been shown to have a significantly higher chance of developing a bacterial coating called a ‘biofilm’, than do smooth implants. It is simply easier for bacteria to grab hold of a textured surface. This biofilm can lead to higher infection and contracture rates. “Biofilm on the implant surface is increasingly understood to be responsible for initiating inflammation, which leads to capsular contracture in the majority of cases.” [In vitro and in vivo investigation of the influence of implants surfaces on the formation of bacterial biofilm in mammary implants. Jacombs A, Tahir S, HuH, Deva A K, Almatroudi A, Wessels W L, Bradshaw D A, Vickery K., Plast Reconstr Surg, 2014 April, 133(4):471e-80e.]. Another paper by similar authors [The role of bacterial biofilms in device-associated infection. Deva A, Adams W, Vickery K., Plast Recontr Surg, November 2013, 1319-1328] outlines strategies and suggests future possible methods to combat the development of implant infection and contracture. This thorough article does not mention the possibility of a composite ePTFE/silicone implant being a viable alternative for decreasing infection and contracture of soft tissue implants. This paper also teaches away by omission from the potential benefits of using a composite ePTFE/silicone implant for optimizing any type of soft tissue implantation.

As described above, there may be benefits of a textured outer surface for decreasing capsular contracture if biointegration of the tissues to the outer rough shell did occur. However, textured implants usually do not have tissue adherence, do have higher infection rates and are associated with an increase incidence of BIA-ALCL.

As described herein and opposite and distinct from the prior art goals, methods, and devices, it can be beneficial to use a solely ePTFE or composite ePTFE and silicone implant for placement in soft tissues for aesthetic, medical, or geolocation purposes. The ePTFE surface can provide a more inert/biocompatible and therefore less reactive surface resulting in less capsular contracture issues, less infection, and better patient acceptability. The ePTFE surface may also encourage a monolayer of cells to grow over the ePTFE nourished by the capsular fluid similar to the situation found in synovial joints and ePTFE aortic grafts. This can provide unique and heretofore unknown benefits over the current state of the art. In this scenario, the implant would be transformed into a ‘living’ implant, thus significantly reducing the reaction of the body as it would recognize the ‘living implant’ as its own. The composite ePTFE/silicone soft tissue implant can also be associated in this manner with decreased infection rates, lighter weight, and fewer palpable ripples and wrinkles.

Additionally, prevent direct bio-integration between an implant and its surrounding tissue. In such cases, creation of an intima, bursa and synovial fluid environment or combinations thereof, in which an implant is not held in rigid position by a capsule and where an implant may or may not have an outer layer of cellular growth can provide unique and heretofore unknown benefits over the current state of the art. In the paper, the role of bacterial biofilms in device-associated infection by Deva A, the following is mentioned: “It is estimated that the cost of revision surgery from implant infection is approaching $1 billion a year in the United States alone. The issue of device-associated infection will continue to grow as our Western population ages and demand for medical prosthetics increases. It is therefore imperative that strategies to reduce the risk of biofilm contamination of medical devices be developed and tested.” [The role of bacterial biofilms in device-associated infection by Deva A et al].

Another paper, published in May 2014 and titled The Pocket Protector: A New e-PTFE Breast Implant Device by Mark Berman, MD, states that “E-PTFE is considered one of the safest synthetic implant materials. Nonetheless, e-PTFE does not have the elastic properties of silicone rubber and would not serve well as a coating on the implant.” Amer. Soc. of Cosmetic Breast Surgery 31^{st} Annual Workshop May 1-4, 2015, which is hereby incorporated in its entirety by reference. As such, the state of the art is that it teaches away from using e-PTFE as a coating on an implant because of its less elastic character than silicone rubber despite its known advantageous properties of being safe.

Further, coupling ePTFE with silicone has always been extremely difficult to achieve due to the very slippery surface characteristics of silicone. Chemical adhesives have not proven reliable in the past but there are some promising new adhesives that might be effective (Cornell University U.S. patent application Ser. No. 14/830,374). Newer methods of linking, adhering or otherwise coupling ePTFE and silicone together can improve bio-acceptance as well as provide numerous other benefits.

Recently, coupling of ePTFE with silicone has been achieved mechanically. 1) Zeus Industrial Products has PCT Publication WO 2014/116490A1 describing how they create penetration of the silicone component into the pores of the electrospun (espun) PTFE component (“Bioweb”, www.zeusinc.com). 2) In addition, Prof. Rainer Adelung's research group at Germany's Kiel University mechanically bonded silicone and PTFE through the use of zinc oxide nanocrystals (ZnO-NC). These crystals are in the shape of a caltrop or tetrapod and essentially nano-staple or anchor the two surfaces together PCT Publication WO 2015/010687A3 and Joining the un-joinable: adhesion between low surface energy polymers using tetrapod ZnO linkers. Jin X, Strueben J, Heepe L, Kovalev A, Mishra Y K, Adelung R, Gorb S N, Staubitz A, Adv Mater, 2012 Nov. 8; 24(42):5676-80]. 3) Implantech corporation currently has ePTFE/silicone composite facial implants on the market to augment and simulate harder tissues such as bone and cartilage (www.implantech.com). Their method of coupling the two materials is unknown.

Thus, as described herein and different from prior methods and soft tissue implant devices, creation of a composite ePTFE (or espun PTFE) and silicone implant for soft tissue placement would be beneficial to the aesthetic, medical and geo-location populations by decreasing capsular contracture problems, decreasing infections, and increasing patient acceptability due to less palpability, lighter weights and fewer overall problems would be beneficial.

In some embodiments, although an internal structure is not silicone gel, each type of implant may have a different structure depending on its purpose. Most are placed in the soft tissue and would benefit from similar characteristics such being softer to touch (minimally palpable), having minimized infection risk, having minimized contracture issues and having minimized displacement issues. They would also benefit from being easy to insert, easy to remove and easy to refill if needed.

Current medical implants generally have a housing made out of silicone, titanium or other similar compositions. They do not utilize an ePTFE housing that, as described above, could minimize infection, scar contracture, displacement, palpability and electrical disturbances (in the case of defibrillators).

The familiar implantable birth control device, known as Norplant, is levonorgestrel housed in a Silastic (silicone) tubing. These are generally placed in the subcutaneous tissue of the volar forearm. It is easy to insert this tubing in this location but it is palpable, has the usual contracture and infection risks of silicone and can get displaced. Implantable defibrillators generally have a very hard titanium housing/shell which is placed in the subcutaneous tissue inferior to the clavicle. Infection, contracture, palpability, tissue displacement and interference of the electrical impulse by the effects of scar on the wires, etc. can take a significant toll.

More biocompatible medical soft tissue tissue implants are needed along with better techniques, devices and methods of insertion and placements of insertion.

As such, there is currently a great need to find an ideal, biocompatible surface for soft tissue implants (aesthetic, medical or geo-location) that a receiving body will universally accept without adverse effects, such as those caused by capsular contracture.

### BRIEF DESCRIPTION OF THE DRAWING(S)

Illustrated in the accompanying drawing(s) is at least one of the best mode embodiments of the present invention In such drawing(s):

FIG. 1A shows a perspective view of a prior art textured round breast implant device.

FIG. 1B shows a perspective view of a prior art smooth round breast implant device.

FIG. 1C shows a top view of an example embodiment of a prior art breast implant.

FIG. 2 shows a top view of a cross section of an example embodiment of a soft tissue implant having a plurality of shell layers.

FIG. 3 shows an exploded cross-sectional view of an example embodiment of a breast implant with ePTFE coating and coupling layer, along the plane indicated by the line 3 in FIG. 2.

FIG. 4A shows a cross-sectional view of an example embodiment of a breast implant with an ePTFE with mechanical adhering.

FIG. 4B shows an example embodiment of a nanostaple device.

FIG. 4C shows an example embodiment of a plurality of nanostaple devices.

FIG. 4D shows an example embodiment of a plurality of nanostaple devices implanted in a surface.

FIG. 5 shows a side cross-sectional view of a fibrous capsule in conjunction with an example embodiment of a breast implant just after implantation and before development of an intima.

FIG. 6 shows an example of a superhydrophobic minimum angle consideration.

FIG. 7 shows a cross sectional view of an example embodiment of a breast implant with an outer ePTFE layer and ePTFE particles implanted within an interior silicone gel.

FIG. 8 shows a cross sectional view of an example embodiment of a breast implant with an outer ePTFE layer and a lattice infrastructure of ePTFE within an interior silicone gel.

FIG. 9 shows an example embodiment where an entire soft tissue implant is comprised of ePTFE.

FIG. 10 shows an example embodiment diagram of pectoralis major and latissimus dorsi muscles.

FIG. 11 shows an example embodiment diagram of gluteal muscles.

FIG. 12 shows an example embodiment diagram of an iliac crest.

FIG. 13 shows an example embodiment diagram of components included in a single rod implant insertion system.

FIG. 14 shows an example embodiment diagram of a soft tissue implant with delivery and retrieval handle.

FIG. 15 shows an example embodiment of a dental implant for bone placement.

### DETAILED DESCRIPTION OF PREFERRED EMBODIMENTS

The following description of the preferred embodiments of the invention is not intended to limit the invention to these preferred embodiments, but rather to enable any person skilled in the art to make and use this invention. Further, the figures herein are not meant to be limiting based on any scale or size relation illustrated but rather are meant to be example embodiments illustrative of concepts. Although any methods, materials, and devices similar or equivalent to those described herein can be used in the practice or testing of embodiments, the preferred methods, materials, and devices are now described.

The present invention relates to improved methods, systems and implant devices for soft tissues having maximal tissue acceptance, including minimal capsular reaction, reduced likelihood of infection and reduced palpability along with other benefits. While these methods, systems and devices are particularly suited for breast implants, it should be understood that the disclosure herein is not limited to such environments and can be used in other soft tissue implantation methods, systems and devices with similar concerns.

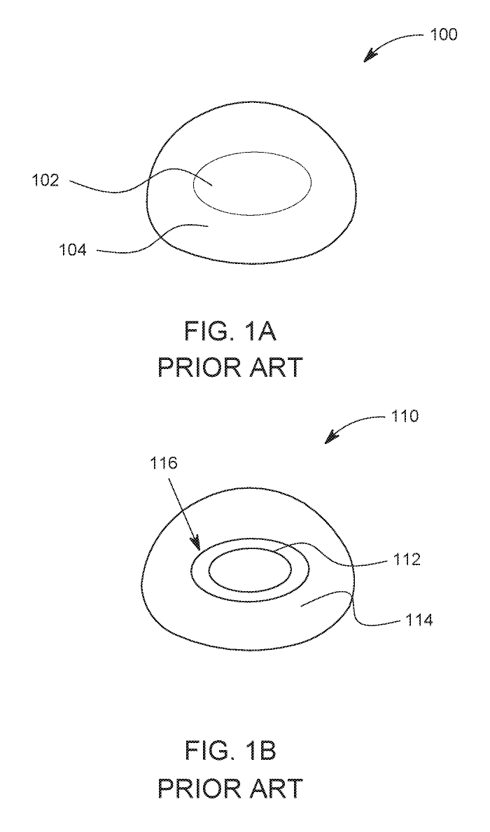

FIG. 1A shows a perspective view of a prior art textured round breast implant device 100. As shown in the example embodiment, prior art textured round breast implant device 100 can include a silicone rubber shell 102 with a textured outer surface 104. In various embodiments silicone rubber shell 102 can be filled with a biologically safe filler substance, obscured by textured outer surface 104 in FIG. 1A. Examples of biologically safe filler substances include silicone gel saline or other substances as currently known in the art.

FIG. 1B shows a perspective view of a prior art smooth round breast implant device 110. As shown in the example embodiment, prior art smooth round breast implant device 110 can include a silicone rubber shell 112 with a smooth outer surface 114. In various embodiments silicone rubber shell 112 can be filled with a biologically safe filler substance 116 as currently known in the art, such as silicone gel, saline or other.

FIG. 1C shows a top cross-sectional view of an example embodiment of a prior art breast implant 120. In the example embodiment, breast implant 120 includes a shell 122 having an outer surface 124 and an inner surface 126. Breast implant 120 can be include a filler substance 128, such as silicone, saline, or others as currently known in the art contained within inner surface 126 of shell 122. As is shown and commonly known, breast implant 120 are generally round but can also be teardrop shaped, having a substantially standard radial diameter 132 from a center point 130 as viewed from a top down perspective. Breast implant 120, when viewed from a side after submuscular implantation (not shown), can create a typically natural teardrop breast contour.

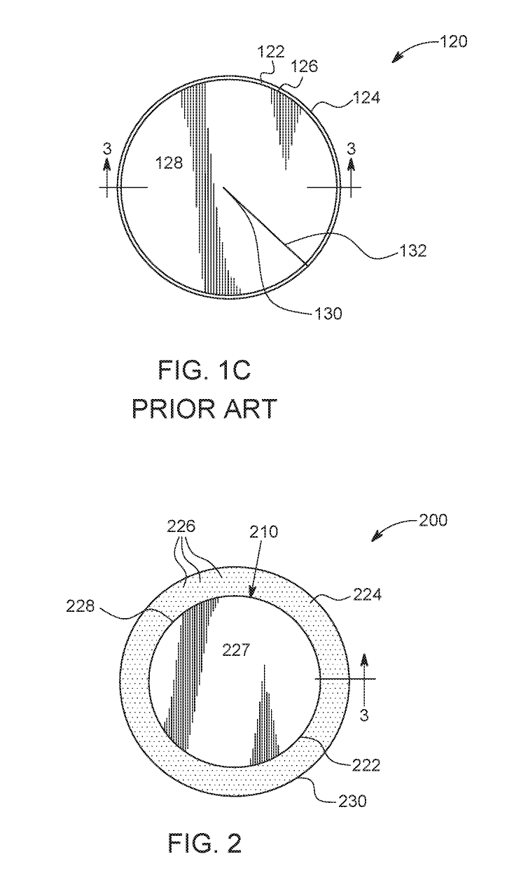

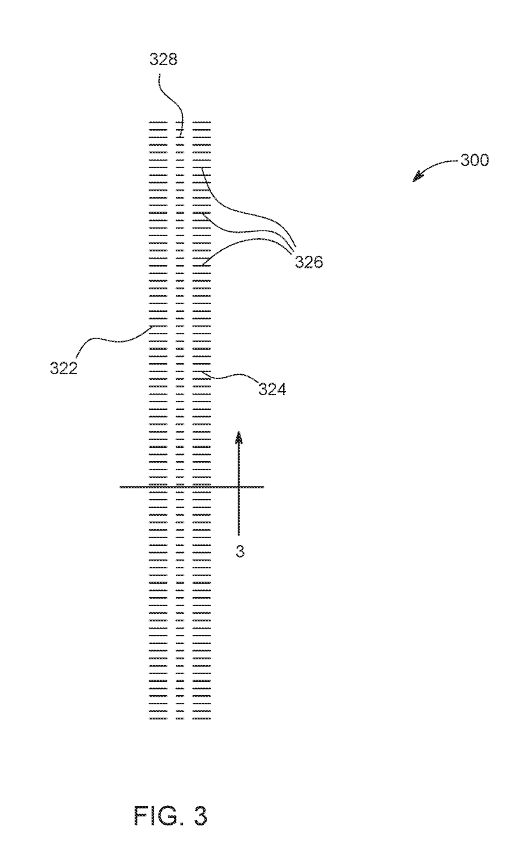

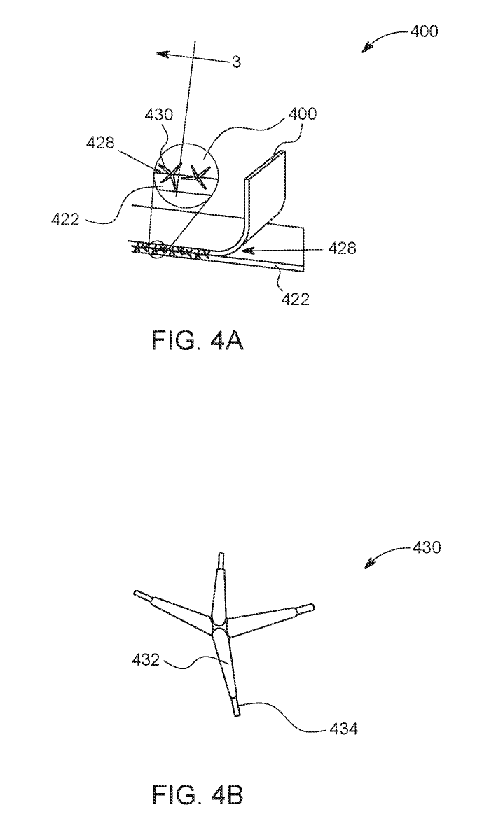

FIG. 2 shows a top view of a cross section of an example embodiment of a soft tissue implant 200 having a plurality of shell layers. In an example embodiment, implant 200 can include a body 210 having a first shell layer 222 that contains a filler substance 227, such as silicone, saline or others known in the art or later developed. First shell layer 222 can be surrounded and coupled with a second shell layer 224. Second shell layer 224 can include ePTFE (expanded polytetraflouroethylene, also known as Gore-Tex® by W. L. Gore & Associates, Inc.) and can include a plurality of micropores 226. An interface 228 between implant first shell layer 222 and and second shell layer 224 can include mechanical (e.g. see FIG. 4A), adhesive (e.g. see FIG. 3) or other coupling means by which second shell layer 224 is coupled, adhered or otherwise connected to first shell layer 222 of body 210 of soft tissue implant 200.

In various embodiments, micropores 226 can one or a plurality of different shapes. These can include regular and irregular shapes. These shapes can have one or a plurality of different dimensions and sizes in different embodiments. For example, in some embodiments, all micropores 226 may have a homogeneous size and shape. In some embodiments, micropores 226 may have homogeneous sizes but a variety of shapes or homogeneous shapes but a variety of sizes. Additionally, micropores 226 may have a regular distribution or irregular distribution in various embodiments.

Use of ePTFE in a second shell layer 224 in an embodiments as a white, soft, lightweight and covered soft tissue implant 200 can be distinguished from the prior art since current breast implants are typically clear, can be firm and do not provide an outer covering. Use of ePTFE as a second shell layer 224 covering an implant 200 can provide mental and psychological benefits for patients contemplating surgery since they may receive peace of mind in feeling a light weight of implant 200 and a soft second shell layer 224 external surface 230 that will be in contact with their internal tissues. The white exterior can also resemble a cloud, further allaying some psychological issues. Additional colors and patterns are also contemplated.

### Coupling ePTFE and Silicone

Connecting, adhering, fastening, joining, or otherwise coupling a first shell layer 222 to a second shell layer 224 at an interface 228 for an implant 200 can be complicated. Where first shell layer 222 can be comprised of silicone rubber, silicone gel or the like and second shell layer 224 can be comprised of at least one sub-layer of ePTFE or Gore-Tex, interface 228 between the first and second shell layers 222, 224 has traditionally been a challenge. However, various new methods, apparatuses, devices and the like are described herein in order to solve these problems. As such, different embodiments of interfaces 228 will be variously described with respect to FIGS. 3-4.

FIG. 3 shows an exploded cross-sectional enlarged view 300 of an example embodiment of an implant interface 328 with a second layer 324 and first layer 322, along the plane indicated by the line 3 in FIG. 2. In the example embodiment, second layer 324 can be an ePTFE layer and first layer 322 can be a silicone layer. Although spaces (not labeled) are shown in FIG. 3 between second layer 324, interface 328 and first layer 322, these are merely to facilitate viewability of the Figure and would be understood by those in the art as being minimal or nonexistent in actual embodiments. Micropores 326 are also shown in second layer 324

In the example embodiment shown in FIG. 3, interface 328 can be one or more layers or mixtures of chemical glues or adhesives can be used to attach, couple or adhere at least one sub-layer of second layer 324 and similar sub-layers of compounds to first layer 322. In some embodiments, interface 328 can be an alloplastic implant material, an example of which is somewhat described by Berman et al. in: “The use of Goretex e-PTFE bonded to silicone rubber as an alloplastic implant material.” Laryngoscope (1986): 96(5), pp4 80-3 which is hereby incorporated in its entirety by reference.

In some embodiments, interface 328 can include a proprietary method, substance, means or mechanism for bonding ePTFE to silicone.

Additional gluing methods and treatments are described in an as yet unpublished Cornell University Alabi technology U.S. patent application Ser. No. 14/830,374, which is hereby incorporated in its entirety by reference. These methods and treatments involve the use of a specialty polymer with orthogonal allyl acrylamide building blocks with R groups that alternately adhere to silicone with other R groups that adhere to ePTFE.

PTFE, also known as Teflon, has some similar characteristics to ePTFE. A method of attaching, bonding or otherwise coupling PTFE (Teflon) to silicone while also reducing problematic issues with seams can be found in the patent application PCT Publication WO 2014/116490 A1, titled “Silicone E-spun PTFE Composites” and filed Jan. 16, 2014, invented by Ballard et al and applied for by Zeus Industrial Products, Inc. of Orangeburg, S.C., which is hereby incorporated in its entirety by reference. As described, electro-spin porous, polymeric components, such as PTFE, can be created around a silicone component and make a composite of PTFE that is adhered to silicone. Another example of a PTFE bonded Silicone which is referred to as ITW Teflon Bonded Silicone®, and described in the Technical Data Sheet “Introducing Patent Pending ITW Teflon Bonded Silicone” was developed by ITW United Silicone of Lancaster, N.Y. and which is hereby incorporated in its entirety by reference.

FIG. 4A shows an exploded cross-sectional view 400 of an example embodiment of a first layer 422 of a soft tissue implant and a second layer 424 with interface 428 showing mechanical coupling elements 430. In the example embodiment, second layer 424 can be an ePTFE coating and first layer 422 can be a silicone layer. As shown, an interface 428 between first and second layers 422, 424 can include a plurality of mechanical coupling elements 430, similarly along an indicated by the line 3 in FIGS. 2-3.

As shown in the example embodiment, mechanical linking of PTFE and Gore-Tex can be accomplished using mechanical coupling elements 430 or otherwise structural components which can have caltrop, tetrapod or other shapes and can have varied orientations in different embodiments. In some embodiments, distribution and orientation of mechanical coupling elements 430 can be standardized or repetitive. Various additional features of mechanical coupling elements 430 are contemplated, including hooks, fasteners, protrusions, and others. In some embodiments both adhesives or adhesive layers and mechanical components 430 can be used or applied at interface 428 and in some embodiments mechanical components 430 can be treated with adhesives.

In various embodiments, mechanical linking of PTFE (Teflon) or ePTFE (Gore-Tex) and silicone can include application of zinc oxide nanocrystals (ZnONC) in the form of caltrops or other materials with caltrop or tetrapod shapes to link opposing surfaces, one having an ePTFE layer and one having a silicone layer. In some embodiments, mechanical components 430 can be placed between ePTFE second layer 424 and a silicone first layer 422 at an interface 428 before applying heat to one or both layers at the same time. This can cause mechanical components 430, also known as microstructure or nanostructure anchors or staples, to embed at least partially into both layers, thus coupling holding the two layers together with respect to each other. These can have metal oxide nano-, micro- or nano-micro-structures which can “join two extremely difficult-to-join polymer layers, namely poly(tertafluorethylene) (PTFE) and cross-linked poly(dimethylsiloxane) (PDMS),” as described in an article by Dodson, published Aug. 26, 2012 on www.gizmag.com titled: “Bringing Teflon and silicone together shows promise for medical applications,” which is hereby incorporated in its entirety by reference. See: http://www.gizmag.com/teflon-silicone-binding/23872/.

In some embodiments, complex shaped metal oxide nano-structures can create interconnected networks that can be applied to surfaces for linking materials. An example is described by Mishra et al in: “Versatile Fabrication of Complex Shaped Metal Oxide Nano-structures and Their Interconnected Networks for Multifunctional Applications,” Kona Powder and Particle J., No. 31, (2014) pp. 92-110, which is hereby incorporated in its entirety by reference.

In some embodiments, adhesion between low surface energy polymers can be accomplished using tetrapodal ZnO components. An example is described by Jin et al in: “Joining the un-joinable: Adhesion between low surface energy polymers using tetrapodal ZnO linkers,” Adv. Mater., Vol. 24, (2012) pp. 5676-5680 which is hereby incorporated in its entirety by reference.

FIG. 4B shows an example embodiment of a nanostaple device 430. Nanostaple device 430 can include a plurality of arms 432 coupled to create an overall structure and may have further components at or near the end 434 of their arms 432.

FIG. 4C shows an example embodiment of a plurality of nanostaple devices 430 in a lattice 440.

FIG. 4D shows an example embodiment of a plurality of nanostaple devices 430 implanted in a surface 450.

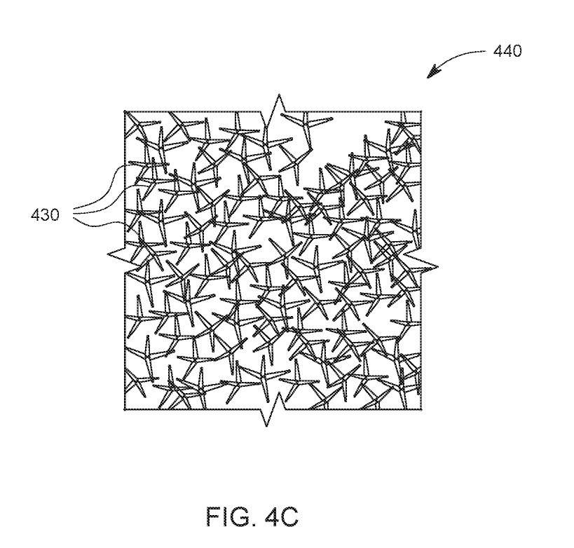

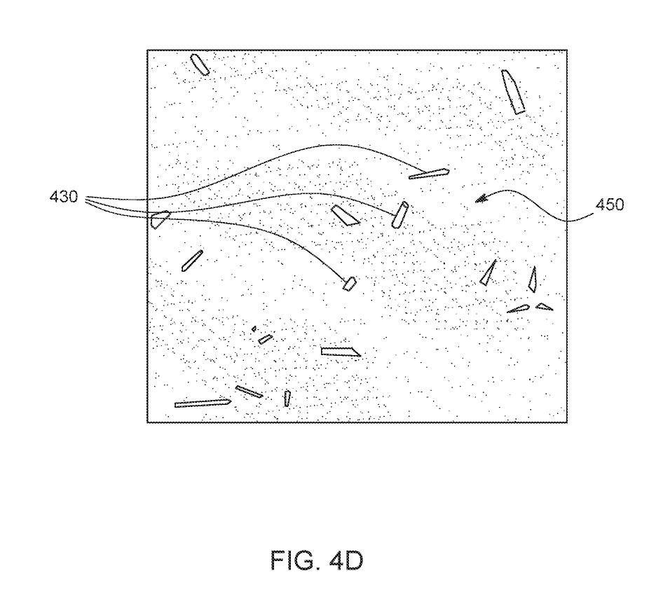

FIG. 5 shows a side cross-sectional view diagram 500 of a fibrous capsule 544 in conjunction with an example embodiment of a soft tissue implant 502 having a second layer 524 that is located exterior to a first layer 522 and coupled via an interface 528. Here, soft tissue implant 502 is a breast implant in a breast 540. As shown, second layer 524 of soft tissue implant 502 can be an ePTFE coating and first layer 522 can be a silicone casing containing a substance 527 just after implantation and before development of an intima. Implant 502 has been surgically implanted below pectoralis muscle 542 and capsule 544 has begun to form around implant 502.

### Benefits and Uses of ePTFE Implants

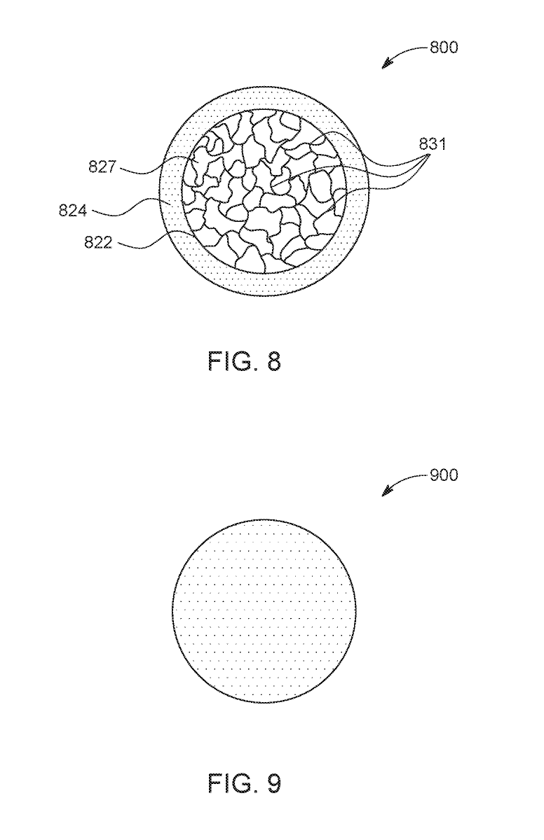

The use of a second layer containing one or more ePTFE sub-layers or similar layers or surfaces as shown in FIGS. 2-5, 7-8, can serve to create a soft, pliable, microporous (e.g. micropores shown in FIGS. 2-3), smooth outer or exterior layer of a soft tissue implant, such as a breast implant. These outer or exterior layers can provide numerous beneficial properties over the prior art as described herein. In some embodiments, an entire soft tissue implant can be comprised of ePTFE (e.g. FIG. 9) as opposed to merely using it as a coating or exterior structure over a different interior structure as shown in FIGS. 2-5, 7-8. In other embodiments (e.g. FIGS. 2-5, 7-8), an ePTFE implant can be an implant with a silicone surface, known in the art as a polysiloxanes implant and filled with a silicone gel or saline, where the silicone surface can be smooth or textured in various embodiments and has a constant or varying thicknesses of an outer layer of ePTFE or similar coating applied to the silicone surface at an interface.

### Micro-Structures and Pre-Treatments

Although smooth to human touch, ePTFE or Gore-Tex has a microporous framework with a porosity of about 10-30 microns, averaging about 22 μm in diameter as described in ePTFE Implants in Rhinoplasty: Literature Review, Operative Techniques, and Outcome, Ham J., Miller P. Facial Plastic Surgery 2003; Vol. 19, No. 4, which is hereby incorporated in its entirety by reference. These microporous framework characteristics, along with non-stick or electronegative and favorable biocompatible properties of ePTFE can help ePTFE resist tissue ingrowth which can be beneficial in various embodiments. Tissue ingrowth can cause tissue adherence to an implant surface of prior art implants and can thwart any postoperative implant movements. However, application of at least one ePTFE coating layer can prevent this tissue adherence, allowing for postoperative implant movements by a surgeon, nurse and patient. These postoperative implant movements can provide numerous benefits, at least one of which is that this can more reliably result in the forming of a post-operative scar capsule with a larger three-dimensional structure than the actual physical three-dimensional size of the implant with the ePTFE coating.

In various embodiments herein, micro-structured gaps, as described above and also referred to herein as micro-pores (e.g. micro-pores 21 in FIGS. 2-3), in ePTFE layers of an ePTFE coated or ePTFE implant can also serve as reservoirs for various chemicals and antibiotics in the exterior surface layer of the ePTFE. These chemicals, along with the soft, smooth characteristic of ePTFE, can serve to optimize biological acceptance of the implant and minimize risk of capsular contracture which will be further described below.

In some embodiments, delivery chemicals or substances implanted, stored or otherwise located in the micro- and nano-structured gaps and pores of a treated ePTFE implant that can be beneficial in assisting a surgeon in implant delivery or other implantation procedures by allowing for and enabling the use of a more slippery or lubricious surface than currently available. This can be accomplished by a manufacturer applied treatment or procedure, by a pre-operative treatment and by a maintenance treatment. These can include surface chemicals or peri-implant space material within the micro-pores of the ePTFE surface. This departs from the current state of the art, which only provides only for secondary treatments to implant surfaces. Pre-treatment or primary treatment of surfaces in this manner can also encourage movement of the implant within the implant capsule and thus provide the benefits of larger post-operative capsule formation.

Additionally or alternatively, in some embodiments, chemicals or other substances can be applied and maintained in the micro-structured gaps and pores that can act to discourage immediate and prolonged tissue adherence. These surface chemicals or peri-implant space materials can function similar to several chemicals, substances and materials which are known in the art but are not integrated within or to an exterior physical surface of an implant. This is not done currently because these materials are merely applied as a secondary treatment to an exterior implant surface. Examples of these secondary treatments of implant surfaces can include: a) a hydrophilic inner layer of a Keller funnel; b) a lubricating material in refresh drops, such as carboxymethylcellulose sodium/glycerin/polysorbate 80; c) a synthetic synovial fluid or d) others. These secondary treatments as referred to herein are applied typically applied to implants at or near the time of implant delivery or implantation. As would be understood in the art, the ePTFE pre-treated or primary treated surface described herein is microporous and can maintain one or more chemicals or substances in addition to or in conjunction with many or all of the secondary treatments in order to provide additional benefits.

In some embodiments, in order to reduce or eliminate capsular contracture and associated morbidity that capsular contracture can cause, additional or alternatively applied surface chemicals or peri-implant space materials can include at least: a) antibiotics, such as Rifampin and others; b) calcium channel blockers, such as Verapamil and others; c) Vitamin E, including the synthetic form alpha-tocopherol; d) Methylprednisolone and others; and e) others. Surface chemicals or peri-implant space materials can also optimize conditions for monocellular adhesion and growth on the outer surface of implants for creation of a ‘living’ layer of cells similar to the intima found in aortic ePTFE grafts and in a synovial joint environment, as opposed to integration by the tissue into the surface of the implant. These chemicals, substances or materials can include one or more of: a) Synovial fluid-like material; b) pre-treatment with alcohol or c) others.

ePTFE reservoirs in the form of micropores can allow chemicals to be layered in and on the ePTFE surface, especially in embodiments where a layer of ePTFE of an implant is relatively thick. In some embodiments this thickness can be from about between one quarter of a millimeter to about two millimeters, while in other embodiments it may be less than or greater than these dimensions. In an example embodiment, an external substance layer can be coated on an outer, external ePTFE implant surface to optimize slippery characteristics of the ePTFE implant as would be beneficial in the first few days or weeks after an implantation procedure. This coating layer can may then dissipate over time and be appropriately absorbed by the body.

In an example embodiment, a secondary or intermediate substance layer can be presoaked by a surgeon or otherwise implanted in the ePTFE micro- or nano-porous reservoirs of the surface layers by a manufacturer, typically prior to a primary or initial external substance layer. In various example embodiments, the secondary or intermediate layer can include an antibiotic layer or inhibitory layer which can serve to prevent or inhibit bacterial infection or infections caused by other biological pathogens. As such, the substances may be activated or begin working at different, appropriate times based on their location.

Similarly, a tertiary layer or other deeper layer or layers for use in treatments can be applied prior to the secondary or intermediate layers and external layers. The tertiary layer or deeper layers can include chemicals, substances and materials which can be expressed, released or administered more slowly, over a longer time period or at a delayed time period. These may assist with cellular adhesion after the initial layers in order to help create a beneficial intima as a bursa or synovial type environment.

Application or implantation of chemical, substance or material layers can be accomplished while accounting for particular timing, interaction, heating, cooling or other chemical, substance or material specific concerns taken into consideration during the pre-treatment or primary treatment process, as would be understood by those in the art.

### Capsular Issues including Tissue Adherence through Contracture and Microbial Growth

A thick or contracting scar capsule around a soft tissue implant can be an undesirable side effect of implantation because it can cause numerous problems including: pain, hardness, and significant distortion of external anatomy. Additionally it can cause electrical disturbance and decreased lifespan of wires associated with internal defibrillators and pacemakers. As such, a soft tissue implant with at least one ePTFE surface can beneficially minimize tissue adherence problems associated prior art implants, including capsular contracture. Microporous ePTFE surfaces can provide smooth, soft and biocompatible surfaces that can move easily in a capsular ‘pocket’ after implantation and thus produce a thin capsule size which can be larger in physical volume size without increased thickness of capsular walls.

One of the main proposed etiologies or medical causes of capsular contracture is microbial contamination. In various embodiments, ePTFE micropores can provide an inhibitory effect on microbial contaminant growth with or without antibiotic soaking and thus can be correlated with lower capsular contracture rates. In an example embodiment, a solution of Fluorocarbons can be applied to an ePTFE layer of an implant and thus inhibit the creation of a biofilm or other undesired bacterial layer or frank infection. Fluorocarbon coated implants have been described by Karlan et al in Potentiation of Infections by Biomaterials: a comparison of three materials. Otolaryngol Head Neck Surg. 1981; 89:528-534, which is hereby incorporated in its entirety by reference, as having a significantly decreased infection rate when compared to silicone.

In some embodiments, ePTFE or Gore-Tex layers can also reduce problems associated with capsular contracture using other mechanisms. Microporosity can be optimized for particular facilitating environments in which the optimized microporous ePTFE layers can allow for topical cellular growth outward or around the ePTFE implant in different amounts and at different rates. This is in direct contrast with ‘tissue integration’ in prior art implants in which the tissue grows into and fixes a location of the implant. Thus, an ePTFE implant can develop a monocellular or multicellular layer (not shown) over the ePTFE surface of the implant. Once this cellular layer is formed, the relationship of the implant with the capsule can perform similarly to performance of naturally occurring biological environments in which a bursa or synovial type environment has two biological membranes opposing each other. In the composite ePTFE and silicone implant embodiments including two biological membranes, one can be a biological membrane capsule and one can be a biological membrane layer around the ePTFE implant. As has been contemplated but heretofore unaccomplished in the art, this type of environment can be a beneficial structural environment: “[i]nterestingly, the macroscopically smooth-surface implant also presents with a rippled microscopic texture on the surface, which might increase the formation of a synovial-type epithelium, experienced in fibrotic breast capsules.” Capsular Contracture by silicone breast implants: possible causes, biocompatibility, and prophylactic strategies, Steiert A E et al, Med Devices (Auckl) 2013; 6:211-218, which is hereby incorporated in its entirety by reference.

These two biological membranes opposing each other can be considered two disconnected structures which are not otherwise rigidly connected or coupled. This differs from the common view in the current state of the art in which textured silicone implants are frequently described as optimally being firmly attached to body tissues, otherwise known as having ‘tissue integration’ or smooth silicone implants result in a synthetic material adjacent to a biological membrane or capsule. Similar layers or intimas can be found along inner lumens of aortic ePTFE or Gore-Tex implants.

As known in various medical arts, creation of an intima or monocellular layer can be described as follows: “[a]s a rule, host cells do not adhere directly to the surface of synthetic implanted materials. Extracellular proteins and proteoglycans form a substrate to which the cells attach. Interactions with cell membrane receptors furnish the linkage for cellular attachment to adsorbed extracellular matrix proteins on implant surfaces. The predominant cells that attach to the protein layer are the fibroblasts. The fibroblasts lay down immature collagen over the matrix on the implant and into the interstices of porous implant. This ingrowth of collagen fibers provides the framework for subsequent cellular adhesion,” as described in: ePTFE Implants in Rhinoplasty: Literature Review, Operative Techniques, and Outcome, Ham J., Miller P. Facial Plastic Surgery 2003; Vol. 19, No. 4, which is hereby incorporated in its entirety by reference. This is different than the prior art teachings in which the actual surrounding tissue is desired or encouraged to grow into or ‘integrate’ into an outside layer of an implant. In some embodiments, some cells can ‘adhere’ or otherwise grow or couple to an exterior ePTFE layer of an implant, forming an essential ‘intima’.

### ePTFE Implants and Silicone Problem Reduction

Additionally, an ePTFE implant can be safer from a medical standpoint for users receiving it as an implant. One or more ePTFE layers adhered to a silicone implant can serve to create an additional barrier to leakage of silicone gel out of a silicone implant when used with a silicone implant. Thus, users can have a reduced chance of negative tissue reaction due to failure of a silicone implant, as compared with traditional silicone implants.

In some embodiments, one or more layers of ePTFE can greatly reduce any penetration of silicone into surrounding tissue since silicone particles are unable to pass through an ePTFE layer because the micropores in an ePTFE layer can be smaller in diameter than the diameter of silicone particles. This can reduce or eliminate problems with silicone particles and silicone-laden macrophages in a capsular environment. Some of these problems are described by Prantl et al, including increased capsular thickness as correlated with an increase in silicone particles and silicone-laden macrophages in a capsule. Capsular Contracture by silicone breast implants: possible causes, biocompatibility, and prophylactic strategies, Steiert A E et al, Med Devices (Auckl) 2013; 6:211-218, which is hereby incorporated in its entirety by reference. Thus, since thickening of a capsule is not desirable in many example embodiments described herein and an ePTFE layer will reduce or inhibit their ability to implant in tissue surrounding the implant, the ePTFE layer can be desirable.

### Other Advantages

An ePTFE or Gore-Tex covering over silicone implants can also provide a more natural ‘feel,’ more similar to a natural breast than current silicone gel implants without ePTFE. This advantage occurs by providing a softer cushion for finger touch due to the soft nature of ePTFE compared to silicone while also minimizing creation and feel of silicone rubber shell undulations, folding and rippling.

ePTFE covered silicone implants can be lighter in weight than current, fully silicone implants of similar size, especially in embodiments where a thicker layer of ePTFE covering is provided. This is due to the fact that the density of ePTFE can be as low as <0.1 with a porosity of 96%, while the density of a silicone gel implant is about 0.97 gm/ml and the specific gravity of saline is 1 gm/ml. Thus, the density of a quantity of ePTFE can be at least 9.7% less dense than a similar quantity of silicone and at least 10% less dense than a similar quantity of saline. The effect of providing implants with ePTFE that are lighter that other implants can make the implants easier to carry for most patients, decrease neck and shoulder pain sometimes associated with heavier breasts due to implants, and decrease undesirable change in implant position, breast ptosis and associated inframammary intertrigo.

Applying hydrophobic (water repellant) nanotechnology to ePTFE (Goretex) or silicone can greatly assist in the biocompatibility of implants covered with this substance, therefore reducing capsular contracture and other risks. An example of how this works is shown in a clothing context at http://silicshirts.com/about-silic-waterproof-shirts/ where Hydrophobic Fabric is described as “The fabric has a nanotechnology bonded to the fibers on the microscopic level. Most liquid molecules will not be able to touch the fabric because of a microscopic layer of air that forms between the liquid and fabric. This is because the fabric is layered with billions of silica particles. Water based liquids will form a 150 degree sphere and roll right off! As a result, this barrier protects your shirt from potential accidents.” It is further described as “Unlike other hydrophobic nanotechnology application processes out there, ours is not cancer-causing or hazardous to your health.”

Neverwet is a superhydrophobic coating made from a proprietary silicon based material that can be used to coat everything from shoes to personal electronics to aircraft. The coating creates surface contact angles of 160-175 degrees; greater than the 150 degrees necessary to deem a substance superhydrophobic.

As applied in the current context with breast implants, the implant can be lighter in weight than a silicone implant without an ePTFE external layer since ePTFE is lighter than standard silicone. Additionally, these implants can have greater biocompatability and less infection than a typical silicone implant without ePTFE external layer.

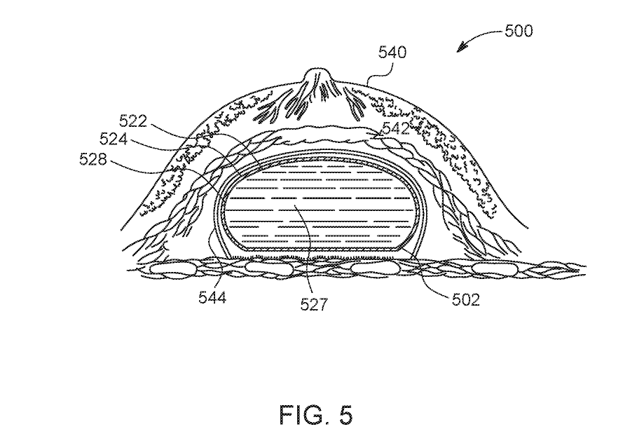

FIG. 7 shows a cross sectional view of an example embodiment of a soft tissue implant with a second layer 724 and an inner layer 722. Second layer 724 can be an outer ePTFE layer. First layer 722 can be a silicone layer coupled with second layer 724 as described herein. First layer can contain a filler substance 727. Within filler substance 727 a plurality of secondary particles 729 can have a different composition than filler substance 727. In some embodiments these filler particles 729 are suspended in fixed positions while in other embodiments, filler particles 729 are free to move. In the example embodiment, these filler particles 729 are ePTFE particles implanted within a filler substance 727 that is silicone gel.

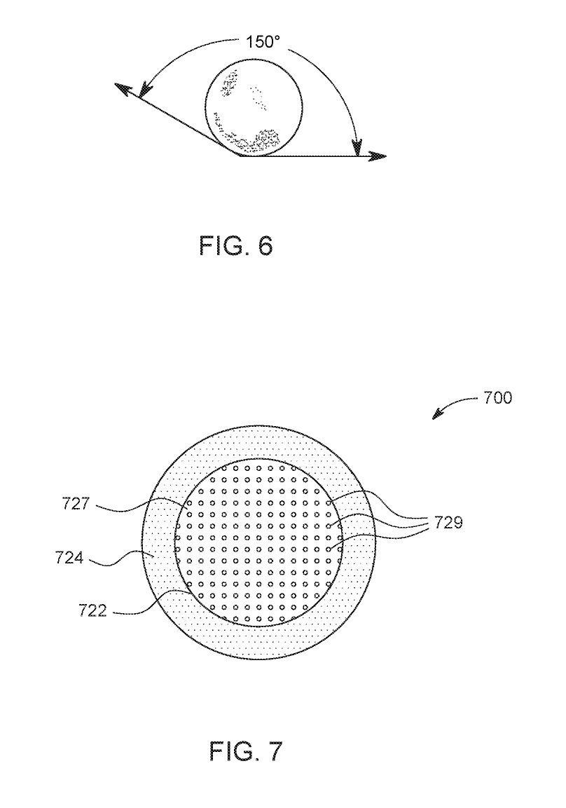

FIG. 8 shows a cross sectional view of an example embodiment of a soft tissue implant 800 with an outer second layer 822 and an inner first layer 724. Second layer 824 can be an outer ePTFE layer. First layer 822 can be a silicone layer coupled with second layer 824 as described herein. First layer can contain a filler substance 827. Within filler substance 827 one or a plurality of strands 831 that can have a different composition than filler substance 827. In some embodiments these strands 831 can be suspended in fixed positions while in other embodiments, strands 831 are free to move.

In the example embodiment, these strands 829 are ePTFE strands implanted within a filler substance 827 that is silicone gel. Second layer 824 can be an ePTFE layer and strands 831 can have the same or different ePTFE qualities from second layer 824. In the example embodiment, strands 831 form a lattice infrastructure of ePTFE within the silicone gel filler substance 827. In various example embodiments, strands 831 that make up lattices may or may not be connected or otherwise coupled with one or more of an inner surface of first shell layer 822, an outer surface of first shell layer 822 or one or more surfaces, layers or sub-layers of a second shell layer 824. As described previously, second shell layer 824 can be an ePTFE layer, while first shell layer 822 can be a silicone rubber shell layer.

Additionally, in some embodiments, particulates, lattices, and combinations thereof of ePTFE matter within a body of a silicone gel core of implant can make the implant lighter in weight as compared to silicone gel cores without these structures. The particulates of ePTFE can be an array of sizes and shapes, both homogeneous and heterogeneous in size and shape in various embodiments. Similarly, three-dimensional lattice structures can also be arranged in a variety of different configurations. These lattices of ePTFE can also be part of an ePTFE shell that goes around the silicone breast implant core. Application of these principles and concepts can improve overall structural integrity of an implant along with decreasing its weight.

As described herein, where ePTFE is used to describe various embodiments it should be understood that espun PTFE and PTFE can be substituted in different embodiments and in different combinations. Also, some embodiments may include combinations and layers may include various sub-layers in different combinations. In some embodiments, pores or other features can include individual coatings and couplings with additional substances and mechanisms.

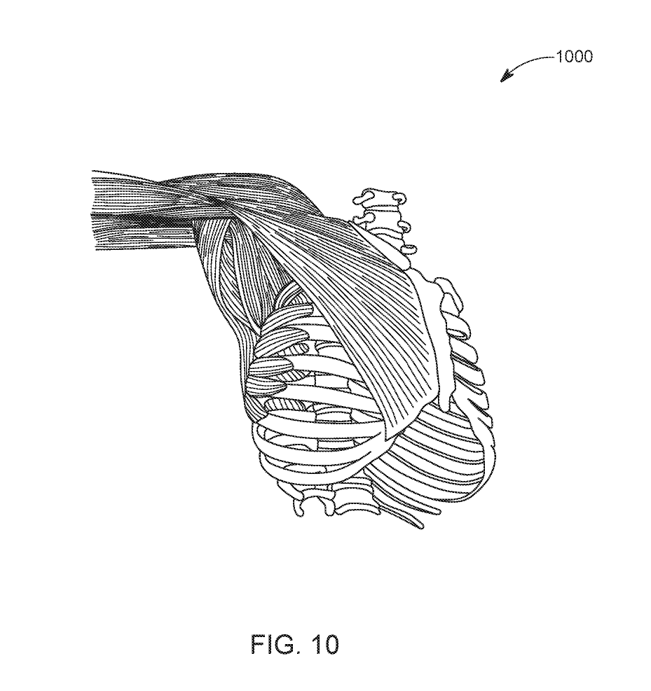

FIG. 10 shows an example embodiment diagram 1000 of pectoralis major and latissimus dorsi muscles.

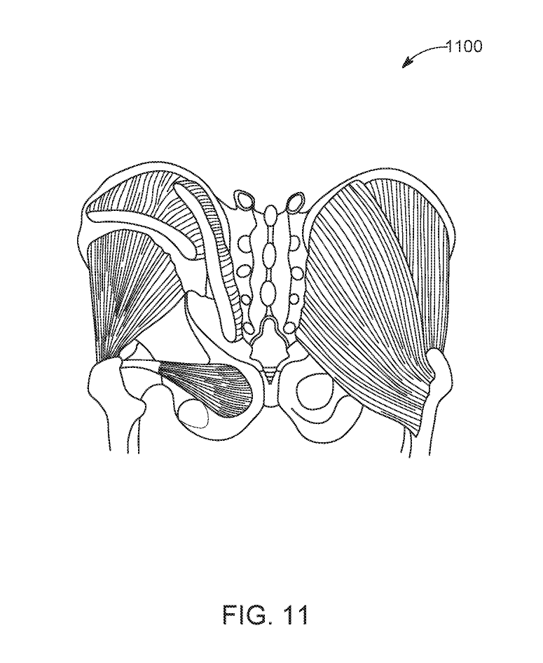

FIG. 11 shows an example embodiment diagram 1100 of gluteal muscles.

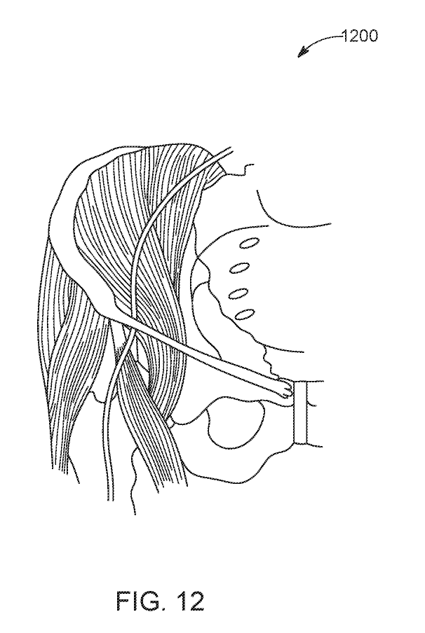

FIG. 12 shows an example embodiment diagram 1200 of an iliac crest.

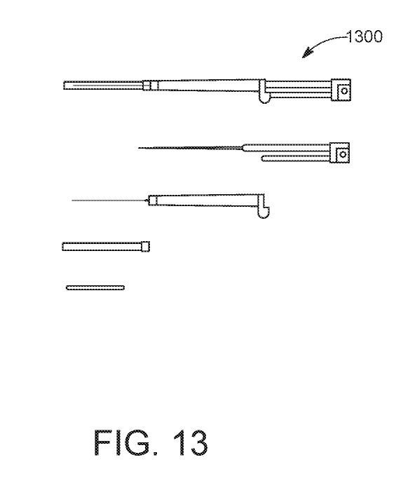

FIG. 13 shows an example embodiment diagram 1300 of components included in a single rod implant insertion system.

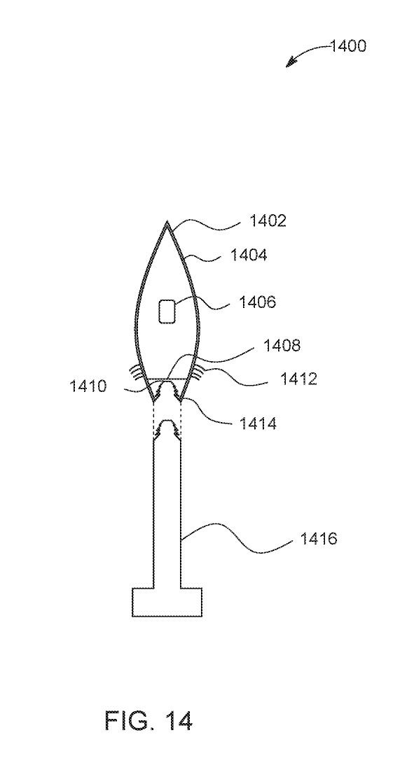

FIG. 14 shows an example embodiment diagram 1400 of a soft tissue implant with delivery and retrieval handle. As shown in the example embodiment, various components are included, such as: a pointed non-cutting tip 1402, an ePTFE outer surface can be squeezed under a front and rear cap 1404, a fenestration for drug emission 1406, a metal backing 1408 can prevent needle tips from movement beyond a desired location, a thick member 1410 can allow needle penetration and seal closed when a needle is withdrawn, quills 1412 can be made of monoccyl or PDS, a small hole 1414 of less than 0.05 mm can be used for tissue ingrown and a handle 1216 for delivery and retrieval.

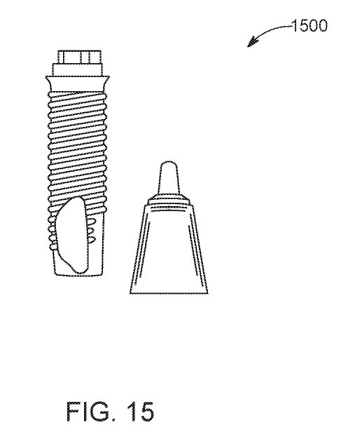

FIG. 15 shows an example embodiment 1500 of a dental implant for bone placement.

Implants for geo-location and identification, medical treatment such as drug delivery or electrical stimulus and monitoring bodily function can be placed anywhere in the body. However, some anatomical areas have not been adequately utilized in the past and may provide new and unique benefits such as providing better concealment, reducing infection risk and infection contraction rates and improved tolerance and acceptance by patients. To elaborate, examples include: 1) Placement into subfascial, submuscular or intramuscular planes along lateral undersurface edge of the pectoralis major muscle, such as in FIG. 10; 2) placement into subfascial, submuscular or intramuscular plane along lateral undersurface edge of the latissimus dorsi muscle such as in FIG. 11; 3) placement in superior buttock tissues, either in a submuscular, supramuscular, between the Gluteus Maximus and Gluteus Minimus muscles, such as in FIG. 12 subfascial or adipose layers; 4) placement within (intraosseous) or medial to the iliac crest, such as in FIG. 13 and others. In general, implant placement near or in muscle provides better insulation from the outside bacterial world, more blood supply to fight any infection and less palpability.

Tubular structures are relatively easy to insert anywhere in the body especially if less than 3 mm in diameter. They can be inserted similar to the methodology suggested for Norplant (or the newer Nexplanon) implants (http://www.arhp.org/publications-and-resources/clinical-proceedings/single-rod/tips), as shown in FIG. 13.

Non-tubular, larger medical implants are best designed in a fusiform, bullet or torpedo (flat or cylindrical) shape so that placement is easier and less bloody. Any configuration that has a non-cutting, pointed tip on it will pass through tissue planes more easily by pushing soft tissues away from its path without cutting tissues. Doctors and surgeons can often use cannulas instead of sharp needles whenever possible to inject fillers in order to decrease possible bleeding. With less bleeding, there is significantly less wound pain, infection, and healing time for patients.

Some medical implants will need to be removed or refilled at different times and these characteristics should also be designed into the medical implant. Easy removal requires easy detection that starts with easy palpation of the implant. When placed into the subcutaneous volar forearm tissues, it is easy to detect. This is beneficial when one wants to know its location but detrimental if the patient does not want another person to inadvertently feel or see the implant. Placing the implant under the edge of the pectoralis major muscle or latissimus major muscle, for example, keeps it from being seen or inadvertently felt. In these positions, it is easily palpated and therefore retrieved or refilled when desired. Other methods of detecting implants include magnetism, radar and dielectric constant changes. Some of these modalities can be brought to a rural medical setting with a smart phone if the implant is appropriately designed (e.g. magnetic component in the end).

Ease of removal and refilling should be designed for particular implants. The current flat or cylindrical bullet-shaped medical implant can be designed with a funneled ‘back’ end to help guide any needle toward the injection port. The conical or flat shaped funnel can have concentric back cut rings that are either circular or spiral. An insertion and removal device can have a complementary conical or flat shaped funnel that has multiple small latches that engage these back-cut concentric lips, essentially locking the delivery/retrieval (DR) handle to the implant. The tip of this DR handle would exactly match and be contiguous with the outside surface of the implant and therefore the implant can be removed with minimal tissue resistance. The tip of the DR handle could also be designed with a screw locking mechanism with the threads of implant backcut and the threads of the DR handle tip being complementary. It can also be designed with a hard plastic tip with lips that engage lips of implant's concentric funneled rear end, such as in FIG. 14.

The outside surface of the implant can also be designed with some very small holes (approximately <0.05 mm) that threads of tissue can grow through to help hold the implant in place, but small enough to break when the implant is retrieved. Implant surfaces can also be coated with absorbable barbed or quill type extensions that can help the hold the implant in place until the material is absorbed. This is about 2-3 weeks for Monocryl (poliglecaprone) and about 2-3 months for PDS (polydioxanone). This barbed material can be manufactured with the ePTFE so that the absorbable quills of Monocryl or PDS extend through the ePTFE.

An ePTFE outer layer can confer on most all these implants a decreased infection rate, decreased capsular contracture rate, greater biocompatability, less palpability and therefore higher patient and doctor acceptance rates.

Intraosseous iliac crest implants that are tubular in shape can be inserted percutaneously, similar to how bone marrow aspirates are performed. Other longer term implants with larger reservoirs can be placed for osseointegration similar to the placement of titanium dental implants (https://en.wikipedia.org/wiki/Dental_implant) [FIG. 14]. A small incision would be placed over the iliac crest and carried down to bone. A small hole is drilled and a hollow cylindrically shaped implant screwed into place. The more superficial end of this implant would have a cap that can be punctured by a needle so that drugs can be placed and the medicine can then be emitted through a fenestration at the tip or anywhere along the side of the osseointegrated implant.

It should also be understood that implants applying the principles and teachings disclosed herein can be used in non-human biological environments, such as by veterinarians or other users on animals.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

The preceding described embodiments of the invention are provided as illustrations and descriptions. They are not intended to limit the invention to precise form described. Other variations and embodiments are possible in light of above teachings, and it is thus intended that the scope of invention not be limited by this Detailed Description, but rather by Claims following.

What is claimed is:

1. An implant apparatus for implantation in a soft-tissue biological environment, comprising:
at least one wall including a primary layer for containing at least one filler substance;
an interface; and
a secondary layer coupled to the primary layer via the interface.

2. The implant apparatus of claim 1, wherein the primary layer further comprises silicone and the secondary layer further comprises ePTFE.

3. The implant apparatus of claim 2, wherein the implant apparatus is a breast implant.

4. The implant apparatus of claim 2, wherein the interface is a mechanical interface.

5. The implant apparatus of claim 4, wherein the mechanical interface further comprises a plurality of mechanical coupling elements.

6. The implant apparatus of claim 5, wherein the mechanical coupling elements have a tetrapod shape.

7. The implant apparatus of claim 2, wherein the interface is an adhesive interface.

8. The implant apparatus of claim 2, further comprising:
the at least one filler substance, comprising:
a silicone gel; and
particulates of a secondary material mixed in the silicone gel.

9. The implant apparatus of claim 8, wherein the particulates are ePTFE particulates.

10. The implant apparatus of claim 1, further comprising:
a lattice structure within the primary layer.

11. The implant apparatus of claim 2, further comprising:
a lattice structure within the primary layer, comprised of at least one ePTFE string.

12. The implant apparatus of claim 11, wherein the lattice structure is coupled to the primary layer, the secondary layer or both.

13. The implant apparatus of claim 11, wherein the lattice structure is not coupled to either the primary layer or the secondary layer.

14. The implant apparatus of claim 1, wherein the secondary layer includes at least one pore for containing a substance.

15. The implant apparatus of claim 14, wherein the substance promotes cellular growth near the implant apparatus.
